(19) United States
(12) Patent Application Publication
(10) Pub. No.: US 2013/0303604 A1
(43) Pub. Date: Nov. 14, 2013

(54) Method of Using Tea Polyphenols for Prevention and Treatment of Malignant Tumors in a Hematopoietic System

(71) Applicants: Li Zhang, Shanghai (CN); Yunhua Liu, Shanghai (CN)

(72) Inventors: Li Zhang, Shanghai (CN); Yunhua Liu, Shanghai (CN)

(21) Appl. No.: 13/941,584

(22) Filed: Jul. 15, 2013

Related U.S. Application Data
(63) Continuation-in-part of application No. 12/921,353, filed on Sep. 7, 2010, now abandoned.

(30) Foreign Application Priority Data
Mar. 6, 2008 (CN) 200810034350.4

(51) Int. Cl. A61K 31/353

(52) U.S. Cl. CPC A61K 31/353
USPC 514/456

(57) ABSTRACT

Tea polyphenols are used to prevent or treat tumors such as leukemia and myeloma, malignant tumors in a hematopoietic system. The tea polyphenols are tea extracts comprising EGCG, EGC, and ECG, or Tegreen97®, a green tea capsule of PHARMANEX, INC.

### CROSS REFERENCE TO RELATED APPLICATIONS

The present patent application is a continuation-in-part of U.S. patent application Ser. No. 12/921,353, filed on Sep. 7, 2010, which, in turn, claims the benefit of priority of China Patent Application Number 200810034350.4, filed on Mar. 6, 2008.

### TECHNICAL FIELD

The invention is related to the use of tea polyphenols or pharmaceutically acceptable salts thereof or any pharmaceutical compositions containing any one of them in preparing medicaments for prevention or treatment of tumors.

### BACKGROUND OF THE INVENTION

Nowadays, the methods for medical treatment of tumors mainly include cytotoxin, induction of apoptosis and differentiation, biological targeted therapy, etc.

Some components originated from natural plants and minerals, such as leurocristine, homoharringtonine, hydroxycamptothecine, paclitaxel and arsenious acid, etc., play a significant role in the first two methods. However, the toxic and side effects, such as the inhibition of proliferation and growth of normal cells in varying degrees, the toxic effects on the peripheral nervous system and the damage to the vital organs, are the important factors which restrict the use of these drugs, and the pharmacoeconomics is also a major factor which cannot be ignored.

The biological targeted therapy uses medicaments which are “custom-made” according to the pathogenesis of tumors including Imatinibmesylate (Imatinib, a tyrosine kinase inhibitor aimed at chronic granulocytic leukemia), rituximab, etc. The price of the medicaments is a large obstacle to the widespread use of those medicaments, and the drug resistance caused by target mutation is a fatal disadvantage.

Because of the above factors, at present, medicaments for treatment of tumors are often used in combination. However, the median lethal dose (LD50) of some medicaments is close to the effective concentration, which easily causes toxic and side effects such as distinct myelosuppression, damage to vital organs, etc, and the tolerance is poor. Also the toxic and side effects of various medicaments have synergistic effects. Therefore the therapeutic effects are only enhanced to some extent, and still a considerable number of patients have drug resistance and show refractoriness and relapse. The limited therapeutic effects of some high bio-technology products are not proportional to their high price. In addition, those products have high preservation requirements and are inconvenient to use. All these factors are major reasons that prompt human beings to keep exploring for new components and new methods to treat tumors.

Tea leaves from Chinese common tea plants are rich in nutriments such as Vitamin C, chlorophyll, carotene, tea polyphenols, etc. Tea polyphenols include catechins, which include epigallocatechingallate (EGCG), epigallocatechin (EGC), epicatechingallate (ECG), and epicatechin (EC). Among them, EGCG is the most abundant one and has the most extensive biological activity, but EGC has stronger anti-oxidant effects than EGCG. Other strong epidemiological evidences also show that an increase in internal concentration of tea polyphenols can provide a powerful protection, eliminate the damage of oxygen free radicals to the body, and have effects on prevention of cancers. Drinking tea has antitoxic and sterilization effects, prevents cancer, and prolongs life. Its healthful effects are well known, but it has not yet been further verified that to what extent and in which aspects the benefits can be.

Further, because catechins in many tea plants are different in their components and contents, and the production process is not standardized, there are at present a variety of edible catechin products and their content and quality are uncertain. They generally belong to the category of health products. The research abroad in inhibiting tumor proliferation using EGCG is mainly in vitro, and the studied cell lines include solid tumor cells such as prostatic cancer, gastric cancer, colon carcinoma, skin cancer, breast cancer, lung cancer, leiomyosarcoma etc. However, its activity of inhibiting tumor proliferation is not very strong and pure EGCG and EGC products are very expensive. Therefore, there is no further report of the in vivo research.

### SUMMARY OF THE INVENTION

In view of the above defects of the prior art, the object of the invention is to provide medicaments for treatment of tumors which are low-cost and convenient to use, and have nearly no toxic and side effects.

The present invention is mainly based on such an idea: although research indicates that catechins like epigallocatechingallate (EGCG) have anti-tumor effects, the price is very high to obtain various pure catechins products through purification of tea polyphenols from plant extracts. If tea polyphenols, which are unpurified and are rich in catechins such as EGCG etc., can be used directly as antitumor medicaments, the production cost will be greatly reduced.

Therefore, the present invention provides uses of tea polyphenols or pharmaceutically acceptable salts thereof or pharmaceutical compositions containing any one of them in preparing medicaments for prevention or treatment of tumors.

The tea polyphenols are tea leave extracts, which include catechins comprising epigallocatechingallate (EGCG), epicatechingallate (ECG) and epigallocatechin (EGC).

In an embodiment of the invention, the pharmaceutical formulations of the medicaments can be any pharmaceutically acceptable formulations such as tablets, capsule, buccal tablets, orally disintegrating tablets, oral fast dissolving tablets, dispersible tablet, masticatory, granules, dry suspension, injection, solution, slow-release formulation, controlled-release formulation, rapid-release formulation, etc.

In an embodiment of the invention, the pharmaceutical compositions also include pharmaceutically acceptable excipients or carriers.

In a preferred embodiment of the invention, the tannin contained in tea leaves is removed from the tea polyphenols, and caffeine, which is an undesirable component, is less than 0.2% by weight in the made formulation.

In an embodiment of the invention, EGCG, ECG and EGC in the tea polyphenols are 50-60%, 20-25% and 8-10% by weight of the catechins, respectively.

In an embodiment of the invention, the capsule is Tegreen97®, a green tea capsule of Pharmnex INC. USA.

The formulation in the invention can be used alone to treat tumors without a combination with other medicaments.

The invention utilizes tea polyphenols, which are tea leave extracts, to prepare antitumor formulations directly instead of pure products of the active components such as

EGCG, ECG and EGC etc. separated from tea polyphenols through complicated and costly purification processes. Thus the production cost of the formulations may be greatly reduced.

Moreover, it is discovered through pharmacological tests in cellular level that tea polyphenols involved in the invention have significant inhibition effects on many kinds of human tumor cells, especially malignant tumor cells in hematopoietic system, and even show good inhibition effects on primary culture cells in some blood tumors. This not only adds a new drug of high-efficiency and low-toxicity for treatment of malignant tumors, but also provides the patients, who suffer from secondary vasculitis after a long-term intravenous infusion of cytotoxin medicaments, with a possibility of keeping being treated in other ways. Through the efficacy test of tumor models in mice, the formulations involved in the invention have significant therapeutical effects on mice tumors. They can reduce the size of the tumors and prolong the survival period of the mice bearing tumor models. Furthermore, the tea polyphenols can be used alone to achieve the above pharmaceutical effects substantially without any other adjuvant therapy for the symptoms.

Moreover, the tea polyphenols involved in this invention hardly have any toxic and side effects of the above-mentioned existing medicaments have. In addition, the plant source of the tea polyphenols is widely spread, and with an abundant supply of the raw materials, and the economic benefit is obvious.

Moreover, the formulations can be capsule, which is very convenient to use and may avoid the damage and stimulation to the tissue and blood vessels caused by injection formulation cytotoxin medicaments which are commonly used before.

Besides, though the tea polyphenols are extracted from the plant, the standardization of the production process can keep the contents of main components of the extracts stable and prevent the difference in contents of components in different batches. Thereby the corresponding research will have good repeatability and comparability. It is a promising product for treatment of tumors. As the formulations for treatment of tumors made of the tea polyphenols do not synergistically increase toxic and side effects when used in combination with other medicaments, and especially have no inhibiting effects on the normal hematopoiesis of the bone marrow, it also provides a promising medicament which can be combined with other medicaments to increase the therapeutic effects together.

Hereinafter, the technical effects of this invention will be further demonstrated with reference to the drawings in order to fully understand the invention.

### DESCRIPTION OF THE DRAWINGS

FIG. 1 is a graph representing proliferation inhibiting effects of tea polyphenols solutions of varying concentrations on a variety of cell lines for 1 day.

FIG. 2 is a graph representing proliferation inhibiting effects of 129.6 μmol/L solution on a variety of cell lines for 1-3 days.

FIG. 3 shows photos of cell morphology observed by a light microscope, representing the effects of tea polyphenols solutions of varying concentrations on morphology of RPM 18226 cell line for 1 day.

FIG. 4a-FIG. 4c are scatter diagrams representing the effects of solutions of varying concentrations on the apoptosis rate of RPMI8226 cell line for 1 day.

FIG. 5a-FIG. 5c are scatter diagrams representing the effects of solutions of varying concentrations on the apoptosis rate of U937 cell line for 2.5 days.

FIG. 6a-FIG. 6d are peak diagrams representing the effects of solutions of varying concentrations on the cell cycle of RPMI8226 cell line for 2 days.

FIG. 7 is a scatter diagram representing the effects of solutions of varying concentrations on the mitochondrial membrane potential of RPMI8226 cell line for different duration.

FIG. 8 shows photos of cell morphology observed by a light microscope, representing the effects of solutions of varying concentrations on morphology of ALL-L2 primary culture cell for 1.5 days.

FIG. 9 is a scatter diagram representing the effects of solutions of varying concentrations on the apoptosis rate of ALL-L2 primary culture cell for 1.5 days.

FIG. 10 shows cell morphology photos of mice tumor tissue sections observed by a microscope.

### DETAILED DESCRIPTION OF THE PREFERRED EMBODIMENTS

The present invention provides a new use of natural tea polyphenols extracted from plants as antitumor formulations. Tea polyphenols involved in the invention are tea leave extractsand are rich in catechins, which includes epigallocatechingallate (EGCG), epicatechingallate (ECG), and epigallocatechin (EGC).

The antitumor formulations made of tea polyphenols can be any pharmaceutically acceptable formulations such as tablets, capsule, buccal tablets, orally disintegrating tablets, oral fast dissolving tablets, dispersible tablet, masticatory, granules, dry suspension, injection, solution, slow-release formulation, controlled-release formulation and rapid-release formulation.

Although the change of content of catechins in tea polyphenols does not affect its antitumor effects significantly, generally, in order to ensure the stability of every batch of products and the comparability of effects, process, in the embodiments of the invention, through the control over the production, the content of catechins is controlled at 60-70% by weight in the antitumor solid formulations made of tea polypheols.

In addition, catechins comprise EGCG, ECG, and EGC. They all have certain antitumor effects. Therefore there are no special restrictions on the proportions of EGCG, ECG and EGC with respect to one another in the tea polyphenols of the invention. Usually, EGCG is the main component among the three. For instance, it contributes more than 50 percent of total weight of the three. EGC is the least. For instance, it contributes about 10 percent or less. However, in order to ensure the stability of every batch of the products and comparability of the effects, through control over production process, EGCG, ECG and EGC are controlled at 50-60%, 20-25% and 8-10% respectively of the total weight of the three in the tea polyphenols of the invention or formulations made of it for treatment of tumors.

To increase the applicability of the formulations made of tea ployphenols described above, during the production of tea polyphenols, undesirable components like tannin and caffeine in the tea leaves can be removed. Moreover, it is better to ensure the content of caffeine is less than 0.5 mg in every 250 mg of the final formulations for treatment of tumors. Almost all people can tolerate such a caffeine level.

The tea polyphenols used in the invention can be made into a variety of pharmaceutical formulations by common pharmaceutical methods, for instance, powder, tablets, capsules or solutions. When in use, the formulations can be taken orally directly or used after dissolving in water.

This mixture formulation of tea polyphenols can well inhibit the proliferation of various tumor cells in vitro. The mechanism may be induction of cell apoptosis through the way of caspase-3, etc. In addition, compared with pure EGCG products and some cytotoxic medicaments, its proliferation inhibition effects are only better. This mixture formulation even has good inhibition effects on some tumor primary culture cells. Taking this tea polyphenols formulation orally can reach the effective antitumor concentration, and more effectively inhibit growth of human multiple myeloma RPMI8226 cell, Lymphoma Jurkat cell, Sudhl-4 cell, etc., inside nude mice.

### Embodiments

The Source of Experimental Material

1. Cell line NB4, NB4-R1, NB4-R2, U937, K562 and RPMI8226 are donated by Ruijin Hospital, School of Medicine of Shanghai Jiaotong University, and Shanghai Institute of Hematology.

Names of the tumor cell lines used herein:

NB4: Human acute promyelocytic Leukemia cell line NB

NB4-R1: Resistant to all-trans retinoic acid human acute promyelocytic Leukemia cell line 1

NB4-R2: Resistant to all-trans retinoic acid human acute promyelocytic Leukemia cell line 2

U937: Human acute monocytic leukemia cell line

K562: Human chronic myelocytic Leukemia cell line

RPMI8226: Human multiple myeloma cell line

2. Experimental mice and nude mice: through Shanghai Jiaotong University, School of Medicine, department of laboratory animal science, purchased from Shanghai SLAC laboratory animal INC. (SCXK (HU) 2003-0003).

3. The formulation for treatment of tumors is made of tea polyphenols from green tea extracts, and the formulation is Tegreen97®, a green tea capsule of Pharmanex INC. USA. There is powder of tea polyphenol in the capsule. The total weight of each capsule is 250 mg, among which catechins are 162 mg (containing 95 mg of EGCG, 37 mg of ECG and 15 mg of EGC, with a molecular weight of 458.4, 442.4 and 306.3, respectively) and the content of caffeine is less than 0.5 mg. (For more detailed components, referring to: Physicians' desk reference®: Ingredients of Tegreen97® Pharmanex, 2005, 59 Edition: 2782.)

The dosage mentioned below is determined by the content of EGCG in Tegreen97®., a green tea capsule, as a reference. Dose of 32.4 μmol/L, 64.8 μmol/L, 129.6 μmol/L and 259.2 μmol/L are respectively equivalent to the supernatant concentration obtained after diluting contents of one capsule by 1:6400, 1:3200, 1:1600, 1:800.

Tea polyphenols solution prepared by Tegreen97®, a green tea capsule, underwent the following antitumor tests:

First, the effects on NB4, NB4-R1, NB4-R2, U937, K562, and RPMI8226 cell lines:

1) Cell growth inhibition test: cells survival rate measured by MTT

2) Wright's stain: observing the morphology change of cell

3) Annexin V-FITC and PI double labeling: quantitative detection of apoptosis rate 4) Cell cycle and analysis of DNA concentration in cell nucleus: revealing possible mechanism of induction of apoptosis

5) Analysis of mitochondrial membrane potential: revealing possible mechanism of induction of apoptosis

Second, the effects on primary culture cell of patients of acute lymphoblastic leukemia and acute monocytic leukemia:

1) Wright's stain: observing the morphology change of the cell 2) Annexin V-FITC and PI double labeling: quantitative detection of apoptosis rate

Third, the therapeutic effects of tea polyphenols solution prepared by Tegreen97®, a green tea capsule, on tumor model in nude mice:

The above tests and results are described in detail as follows.

First, the effects on NB4, NB4-R1, NB4-R2, U937, K562, and RPMI8226 cell lines.

1) Cell Growth Inhibition Test: Cell Survival Rate Measured by MTT

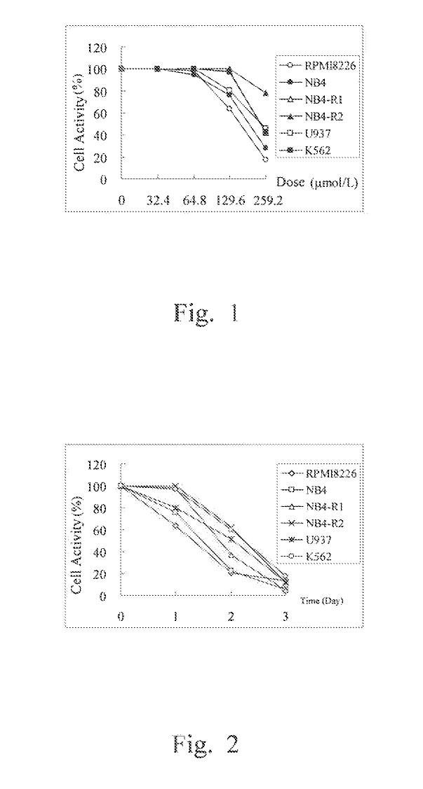

The curve in FIG. 1 shows proliferation inhibiting effects of tea polyphenols of Tegreen97® capsule in the dosages of 32.4 μmol/L, 64.8 μmol/L, 129.6 μmol/L, and 259.2 μmol/L on a variety of cell lines for the duration of 1 day, which are 162, 324, 648 and 1296 μmoles per five liters of blood.

The curve in FIG. 2 shows proliferation inhibiting effects of 129.6 μmol/L dosage on a variety of cell lines for the duration of 1-3 days.

The results indicate that there are significant proliferation inhibition effects on each cell line in the dosage of 129.6 and 259.2 μmol/L for the duration of 2-3 days.

2) Wright's Stain: Observing the Morphology Change of Cell Apoptosis

Through Wright's stain, the morphology change of cell apoptosis is observed by the light microscope at 100 times.

As shown in FIG. 3, after the tea polyphenols solution of varying concentrations acted on multiple myeloma cell line RPMI8226 for 1 day, the control group (0 μmol/L) shows evenly lightly stained cell nuclei, loose chromatin and normal proportion of cytoplasmic; after 32.4 μmol/L, 64.8 μmol/L, 129.6 μmol/L, and 259.2 μmol/L of tea polyphenols acted on RPMI8226 cell for 1 day, membrane shrinkage, chromatin condensation, pyknotic nucleus and apoptosis body appeared.

The results indicate tea polyphenols can induce apoptosis of RPMI8226 cell line, and the higher the concentration is, the more obvious the effects are.

3) Annexin V-FITC and PI Double Labeling: Quantitative Detection of Apoptosis Rate

Multiple Myeloma Cell Line RPMI8226

### Quantitative Detection of Apoptosis Rate by Flow Cytometry (Annexin V-FITC and PI Double Labeling Method)

FITC, Annexin-V labeled by fluorescein isothiocyannate, combined with externalized membrane phosphatidylserine (PS) when apoptosis happened. Apoptotic cells showed Annexin-V positive.

PI, propidium iodide, combined with nucleic acid. Apoptotic cells showed PI positive.

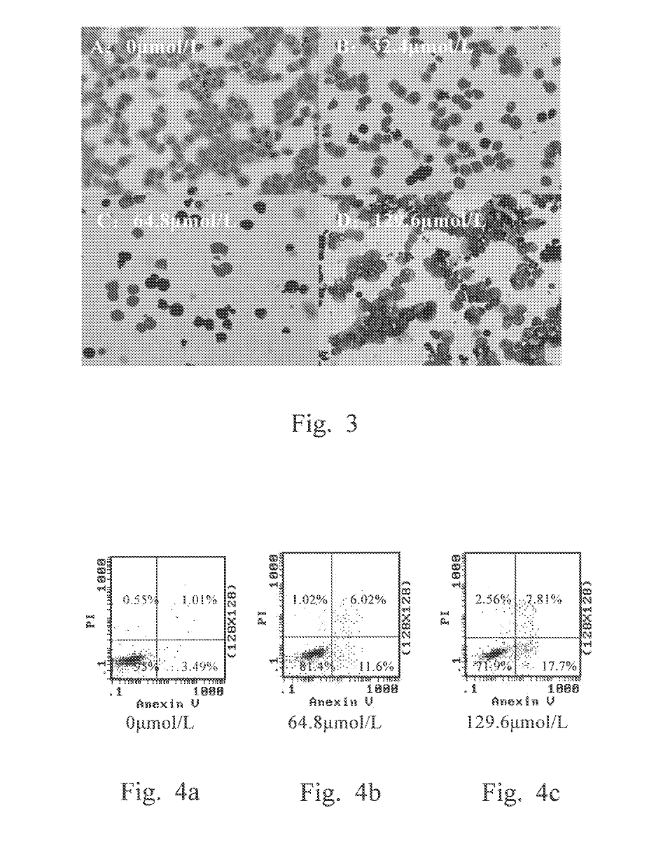

FIG. 4a-FIG. 4c are fluorescence scatter diagrams indicated by flow cytometry. In the diagrams, the lower left quadrant shows Annexin-V FITC and PI double-labeled negative cells (normal cells); the lower right quadrant shows Annexin-V FITC single-labeled positive cells (early apoptotic cells); the upper right quadrant shows Annexin-V FITC and PI double-labeled positive cells (late apoptotic cells); the upper left quadrant shows PI single-labeled positive cells (dead cells).

The results indicate that, after tea polyphenols of varying concentrations acted on multiple myeloma cell line RPMI8226 for 1 day, in control group (0 μmol/L) normal cells are 95%. After tea polyphenols of concentrations of 64.8 μmol/L and 129.6 μmol/L acted on RPMI8226 cell line for 1 day, early apoptotic and late apoptotic cells increased obviously.

Human Acute Monocytic Leukemia Cell Line U937

### Quantitative Detection of Apoptosis Rate by Flow Cytometry (Annexin V-FITC and PI Double Labeling Method)

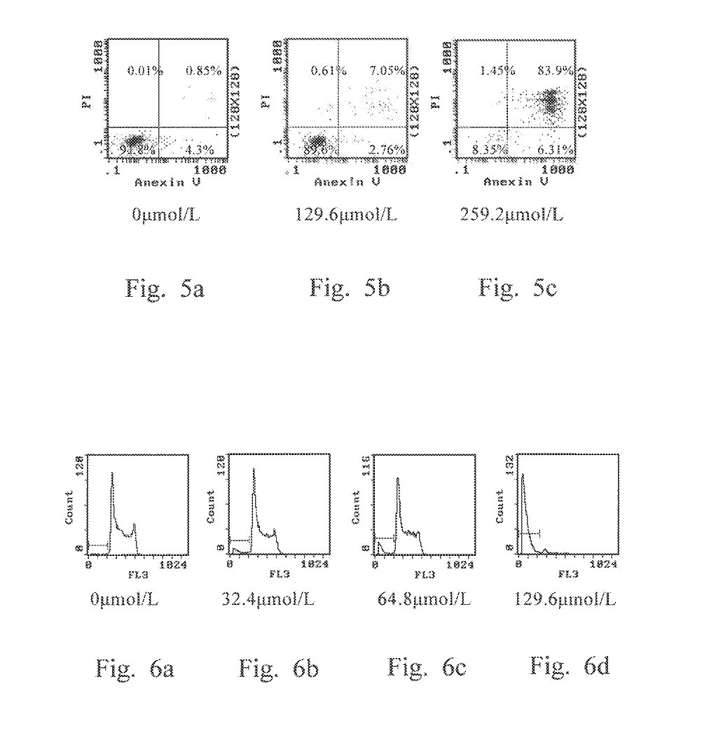

FIG. 5a-FIG. 5c are fluorescence scatter diagrams indicated by the flow cytometry. In the diagrams, the lower left quadrant shows Annexin-V FITC and PI double-labeled negative cells (normal cells); the lower right quadrant shows Annexin-V FITC single-labeled positive cells (early apoptotic cells); the upper right quadrant shows Annexin-V FITC and PI double-labeled positive cells (late apoptotic cells); the upper left quadrant shows PI single-labeled positive cells (dead cells).

The results indicate that, after tea polyphenols of varying concentrations acted on human acute monocytic leukemia cell line U937 for 2.5 days, in control group (0 μmol/L) normal cells are 94.7%. After tea polyphenols of concentrations of 129.6 μmol/L and 259.2 μmol/L acted on RPMI8226 cell line for 2.5 days, early apoptotic and late apoptotic cells increased obviously.

4) Cell Cycle and Analysis of DNA Concentration in Nucleus: Revealing Possible Mechanism of Induction of Apoptosis

Measured by the flow cytometry, after solutions of varying concentration acted on human multiple myeloma cell line RPMI8226 for 2 days, there was no significant difference in the proportions in the G1, S, G2 stages (which are 32%, 64.4% and 3.5%, respectively).

FIG. 6a-FIG. 6d are peak-shaped diagrams indicated by the flow cytometry. In the diagrams, FL3 and count represent the percentage of hypodiploid cells, which are an indicator of apoptosis. By analysis of the DNA content in the nucleus, with the increase of the concentration, the percentage of hypodiploid cells also increased (in control group, after tea polyphenols of concentration of 32.4 μmol/L, 64.8 μmol/L and 129.6 μmol/L acting for 2 days, the percentages of hypodiploid cells were 0.5%, 8.3%, 15.7% and 94.1%, respectively).

The results indicate that tea polyphenols inhibit the proliferation of human multiple myeloma cell line by the means of inducing apoptosis, which correlates to the concentrations.

5) Analysis of Mitochondrial Membrane Potential: Revealing Possible Mechanism of Induction of Apoptosis

By means of the flow cytometry (Rhodamine Rh123 fluorescent labeling method), mitochondrial membrane potentials inside the cells were quantitatively detected to explore possible mechanism of proliferation inhibition.

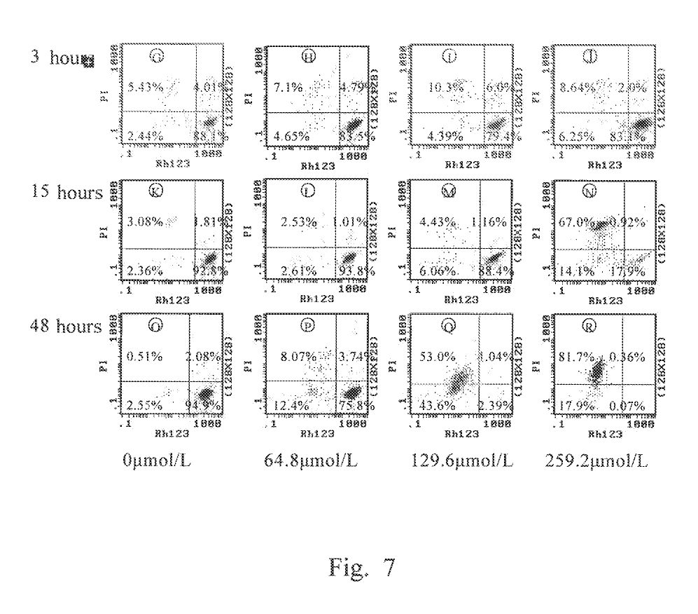

G-R in FIG. 7 provide conditions about the proliferation and apoptosis of human multiple myeloma cell line under different dosages and different acting time periods. In comparison with the control group (0 μmol/L), after 64.8 μmol/L, 129.6 μmol/L, and 259.2 μmol/L tea polyphenols acted on human multiple myeloma cell line for 3 hours, mitochondrial membrane potential began to decline and was in a time-dependant manner.

The results indicate that the mechanism of tea polyphenols for inhibiting the proliferation of human multiple myeloma cell line is relevant to induction of apoptosis and may be relevant to the damage on mitochondria.

Second, the effects on primary culture cells of patients of acute lymphoblastic leukemia and acute monocytic leukemia:

1) Wright's Stain: Observing Morphology Change of Cells

Through Wright's stain, morphology change of cells is observed.

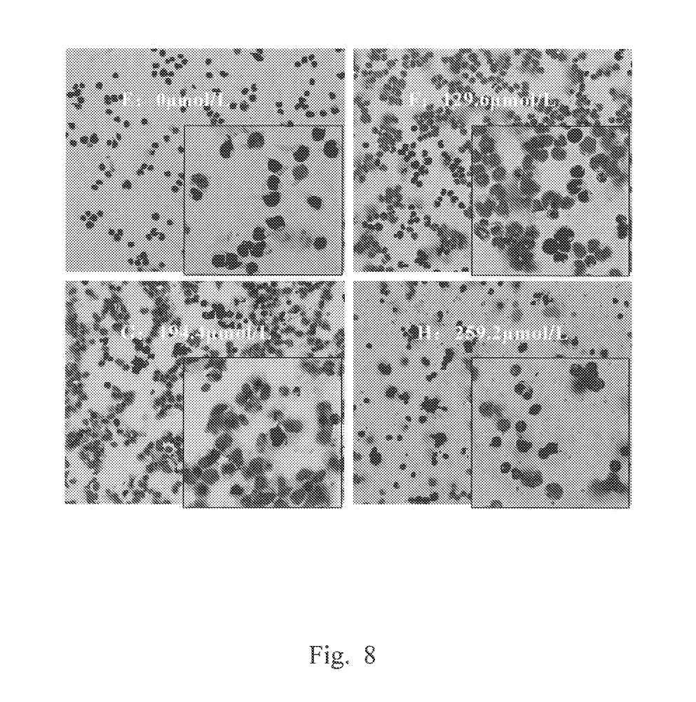

As shown in FIG. 8, after tea polyphenols of varying concentrations acted on primary culture cells of patients of acute lymphoblastic leukemia for 1.5 days, the control group (0 μmol/L) showed evenly lightly stained cell nuclei, loose chromatin and normal proportion of cytoplasmic; after tea polyphenols of concentrations of 129.6 μmol/L, 194.4 μmol/L and 259.2 μmol/L acting for 1.5 days, membrane shrinkage, chromatin condensation, pyknotic nucleus and eosinophilic body (apoptosis body) appeared in primary culture cells.

The results indicate that tea polyphenols can induce apoptosis of primary culture cells of patients of acute lymphoblastic leukemia, and the higher the concentration is, the more obvious the effects are.

2) Annexin V-FITC and PI Double Labeling: Quantitative Detection of Apoptosis

### Quantitative Detection of Apoptosis Rate is Performed by the Flow Cytometry (Annexin V-FITC and PI Double-Labeling Method)

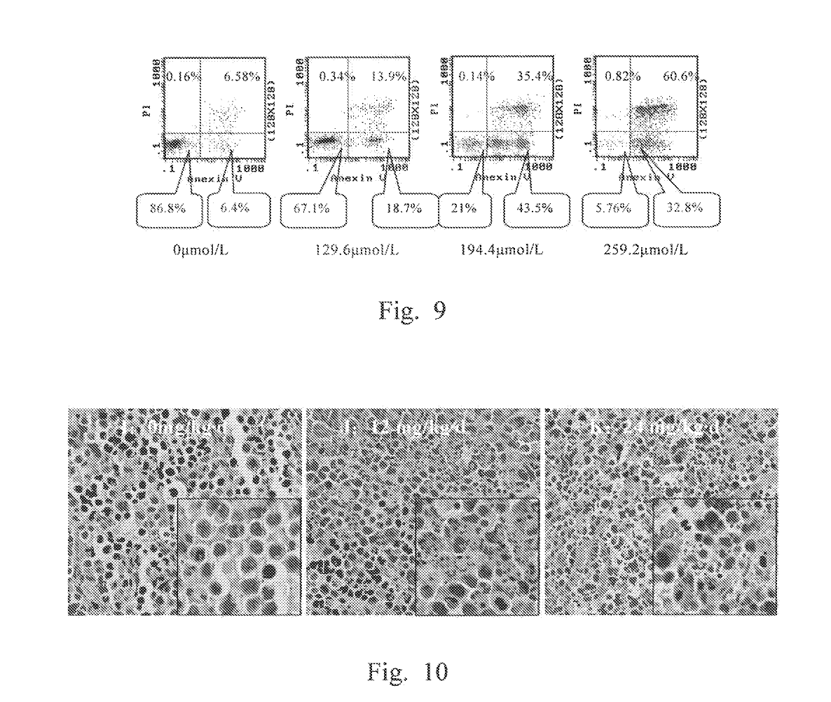

As shown in FIG. 9, in the scatter diagram, the lower left quadrant shows Annexin-V FITC and PI double-labeled negative cells (normal cells); the lower right quadrant shows Annexin-V FITC single-labeled positive cells (early apoptotic cells), the upper right quadrant shows Annexin-V FITC and PI double-labeled positive cells (late apoptotic cells), the upper left quadrant shows PI single-labeled positive cells (apoptotic cells).

The results indicate that, after tea polyphenols of varying concentrations acted on acute lymphoblastic leukemia primary culture cells for 1.5 days, in control group (0 μmol/L) normal cells were 86.8%. After tea polyphenols of concentrations of 129.6 μmol/L, 194.4 μmol/L and 259.2 μmol/L acting for 1.5 days, early apoptotic and late apoptotic cells increased obviously, while normal proliferating cells decreased significantly which are 67.1%, 21% and 5.76%, respectively.

Third, the therapeutic effects of tea polyphenols solution prepared by Tegreen97®, a green tea capsule, on tumor model in nude mice:

RPMI8226 cell line of 4×10⁸ cells per ml was prepared, and each mouse was injected 1.5 ml under back skin. Tumors appeared after about 10 days. From the 16th day after injection, the supernatant of capsule tea polyphenols solution was administered to the mice orally for 5 consecutive days, the concentration of EGCG was 48 mg/ml and it was taken orally once per day. Physiological saline was administered to the control group. From the 16th day, the tumor sizes were measured every day. See results in the following table.

| | Size of the lump (cm × cm) | | | | | | |
|---|---|---|---|---|---|---|---|
| Feeding | 16^{th} days after | | | | | 21^{st} | 22^{nd} |
| dosage | injection | 17^{th} day | 18^{th} day | 19^{th} day | 20^{th} day | day | day |
| 24 mg/kg · d | 1.06 × 0.96 | 0.952 × 0.69 | 0.73 × 0.344 | 0.392 × 0.34 | 0.28 × 0.04 | | Dead |
| 12 mg/kg · d | 0.786 × 0.756 | 0.76 × 0.70 | 0.68 × 0.54 | 0.55 × 0.52 | 0.38 × 0.22 | Dead | |
| Control | 0.51 × 0.44 | 0.84 × 0.76 | Dead | | | | |
| group | | | | | | | |

In the control group, the volume of tumor kept increasing after the 16^{th} day and the mice died on the 18th day. In the treated group, since the medicament was administered from the 16^{th} day, the tumor continually shrunk instead of growing and the dying time of mice was delayed for at least 3-4 days in comparison with the control group. This indicates tea polyphenols have significant antitumor effects.

The tumor tissues of mice, which were fed with different dosages of tea polyphenols, were taken out, and then made into paraffin sections which were dyed by HE, and then the morphology change of the cells was observed by the microscope.

As shown in FIG. 10, the tumor tissue of mice which are fed with 0 mg/kgd tea polyphenols shows evenly lightly stained cell nuclei, loose chromatin and normal proportion of cytoplasmic; after the mice were fed with 12 mg/kgd and 24 mg/kgd tea polyphenols, membrane shrinkage, chromatin condensation, pyknotic nucleus and eosinophilic body (apoptosis body) appeared in tumor cells.

The results indicate that tea polyphenols can induce apoptosis of tumor cells in mice, and the higher the concentration is, the more obvious the effects are.

### Formulas 1-6:

EGCG, ECG, EGC, Chlorophyll a purchased from Shanghai Winherb Medical Science Co., Ltd., Carotene purchased from Kamai Shu Ciotechnology (Shanghai) Co., Ltd., Vitamin-C purchased from Double Crane Pharmaceutical Co., Ltd. (Beijing, China) and RPMI 1640 culture medium purchased from Life technologies corporation (US) were used to prepare the following preparations:

1. EGCG 50 mg+5 mL RPMI 1640 to form a preparation solution of 10 mg/mL; 1 mL of the preparation solution+9 mL RPMI 1640 to form a drug solution of 1 mg/mL (equivalent of 2.2 mM).

2. EGC 50 mg+5 mL RPMI 1640 to form a preparation solution of 10 mg/mL; 1 mL of the preparation solution+9 mL RPMI 1640 to form a drug solution of 1 mg/mL;

3. ECG 50 mg+5mL RPMI 1640 to form a preparation solution of 10 mg/mL; 1 mL of the preparation solution+9 mL RPMI 1640 to form a drug solution of 1 mg/mL;

4. EC 50 mg+5mL RPMI1640 to form a preparation solution of 10 mg/mL; 1 mL of the preparation solution+9 mL RPMI 1640 to form a drug solution of 1 mg/mL;

5. Chlorophyll a 1 mg+1 mL RPMI 1640 to form a drug solution of 1 mg/mL;

6. Carotene 10 mg+10 mL RPMI 1640 to form a drug solution of 1 mg/mL; 7. 1 mL of Vitamin-C of 100 mg/mL+9 mL RPMI 1640 to form a preparation solution of 10 mg/mL; 1 mL of the preparation solution+9 mL RPMI 1640 to form a drug solution of 1 mg/mL.

The drug solutions of 1 mg/mL prepared above were mixed according to the milliliters shown in Table 1 below, and to the resulting mixed solutions RPMI 1640 was added to a total volume of 5 mL.

TABLE 1

| | Volume (milliliters) | | | | | | |
|---|---|---|---|---|---|---|---|
| | | | | Chloro- | | | |
| Formula | EGCG | ECG | EGC | phyll a | Vitamin-C | Carotene | EC |
| 1 | 0.919 | 0.376 | 0.134 | 0.05 | 0.192 | | |
| 2 | 0.919 | 0.376 | 0.167 | 0.05 | | 0.159 | |
| 3 | 0.919 | 0.334 | 0.15 | | | 0.134 | 0.134 |
| 4 | 0.919 | 0.418 | 0.15 | | 0.084 | 0.1 | |
| 5 | 0.835 | 0.376 | 0.15 | 0.084 | | | 0.225 |
| 6 | 1.002 | 0.376 | 0.15 | | 0.067 | | 0.075 |

The tumor cell growth inhibition rates of the formulas 1-6 so prepared were tested according to the method as follows:

1. NB4 cell line of 5×10⁵/mL was prepared with RPMI 1640 containing 20% FBS (fetal bovine serum, purchased from Life technologies Corp., US), which was injected into cells of a 96-cell plate with 100 μL/cell;

2. The formulas 1-6 were diluted so that a concentration of EGCG therein is equal to 0.05 mM (equivalent to 250 μmoles per 5 L of blood), which was injected into the cells of the 96-cell plate with 100 μL/cell;

3. The 96-cell plate was placed in a culture box at 37° C. and an atmosphere of 5% CO₂ for incubation for a period of one day, two days and three days;

4. 5% MTT was injected to each cell with a dosage of 20 μL/cell four hours before the end of the incubation, and then incubation was resumed at 37° C. and an atmosphere of 5% CO₂ for four hours;

5. The incubation mixture was centrifuged at 2000 rpm for five minutes, and washed with PBS three times after the supernatant was disregarded by suction;

6. To each cell 200 μL of DMSO was added, and vibrated at room temperature for 15 minutes;

7. The absorbance of the solution at wavelength of 570 nm was carried out by a spectrophotometer. The inhibiting rate was calculated=1−(OD of the formula group/OD of the control group).

The results are shown in Table 2, which indicates that the formulas having concentrations of EGCG of 50-60%, ECG of 20-25%, and EGC of 8-10% with chlorophyll, carotene or Vitamin-C show a significant inhibition effect on the tumor cell growth.

TABLE 2

| | | |
|---|---|---|
| | Percentage of | Inhibiting rate on |
| Formula | main components | tumor cell growth |

| | | | |
|---|---|---|---|
| 1 | EGCG | 55% | 1 Day: 82.5% |
| | ECG | 22.5% | 2 Days: 89.5% |
| | EGC | 8% | 3 Days: 87.7% |
| | Chlorophyll | 6.5% | |
| | Vitamin-C | 8% | |
| 2 | EGCG | 55% | 1 Day: 68.2% |
| | ECG | 22.5% | 2 Days: 84.9% |
| | EGC | 10% | 3 Days: 88.4% |
| | Chlorophyll | 6.5% | |
| | Carotene | 6% | |
| 3 | EGCG | 55% | 1 Day: 83.4% |
| | ECG | 20% | 2 Days: 89.8% |
| | EGC | 9% | 3 Days: 89.4% |
| | Carotene | 8% | |
| | Epicatechin (EC | 8% | |
| 4 | EGCG | 55% | 1 Day: 86.7% |
| | ECG | 25% | 2 Days: 90.6% |
| | EGC | 8% | 3 Days: 89.8% |
| | Vitamin-C | 5% | |
| | Carotene | 6% | |
| 5 | EGCG | 50% | 1 Day: 60.8% |
| | ECG | 22.5% | 2 Days: 84.4% |
| | EGC | 9% | 3 Days: 87.2% |
| | Chlorophyll | 10% | |
| | Epicatechin (EC) | 8.5% | |
| 6 | EGCG | 60% | 1 Day: 85.0% |
| | ECG | 22.5% | 2 Days: 90.3% |
| | EGC | 9% | 3 Days: 89.3% |
| | Vitamin-C | 4% | |
| | Epicatechin (EC) | 4.5% | |

Various aspect of the present invention includes (but not limited to) the oloowings:

1. A method of using tea polyphenols for prevention or treatment of a malignant tumor in a hematopoietic system comprising administering to a subject in need thereof an effective amount of tea polyphenols.

2. The method of aspect 1 wherein said tea polyphenols comprise catechins

3. The method of aspect 2, wherein said catechins comprises epigallocatechingallate, epicatechingallate and pigallocatechin.

4. The method of aspect 3, wherein said subject has a concentration of catechins in blood thereof not lower than 129.6 μmol/L as a result of said administering.

5. The method of aspect 1, wherein a composition comprising said tea polyphenols is administered to said subject, so that said tea polyphenols are administered to said subject, wherein said composition comprises 50-60% epigallocatechingallate, 20-25% epicatechingallate and 8-10% epigallocatechin.

6. The method of aspect 5, wherein said composition comprises an extract from tea leaves.

7. The method of aspect 6, wherein said extract contains less than 0.2% of caffeine by weight of the extract.

8. The method of aspect 6, wherein said extract is substantially free of tannin.

9. The method of aspect 2, wherein said tea polyphenols comprise 60-70% of catechins by weight of the tea polyphenols.

10. The method of aspect 5, wherein said composition further comprises 4-8% Vitamin-C based on the weight of the composition.

11. The method of aspect 1, wherein said malignant tumor is leukemia.

12. The method of aspect 1, wherein said malignant tumor is myeloma.

From the above description, one skilled in the art can easily ascertain the essential characteristics of the present invention, and without departing from the spirit and scope thereof, can make various changes and modifications of the invention to adapt it to various usages and conditions. Thus, other embodiments are also within the scope of the following claims.

1. A method of using tea polyphenols for prevention or treatment of a malignant tumor in a hematopoietic system comprising administering to a subject in need thereof an effective amount of tea polyphenols.

2. The method of claim 1 wherein said tea polyphenols comprise catechins

3. The method of claim 2, wherein said catechins comprises epigallocatechingallate, epicatechingallate and pigallocatechin.

4. The method of claim 3, wherein said subject has a concentration of catechins in blood thereof not lower than 129.6 μmol/L as a result of said administering.

5. The method of claim 1, wherein a composition comprising said tea polyphenols is administered to said subject, so that said tea polyphenols are administered to said subject, wherein said composition comprises 50-60% epigallocatechingallate, 20-25% epicatechingallate and 8-10% epigallocatechin.

6. The method of claim 5, wherein said composition comprises an extract from tea leaves.

7. The method of claim 6, wherein said extract contains less than 0.2% of caffeine by weight of the extract.

8. The method of claim 6, wherein said extract is substantially free of tannin.

9. The method of claim 2, wherein said tea polyphenols comprise 60-70% of catechins by weight of the tea polyphenols.

10. The method of claim 5, wherein said composition further comprises 4-8% Vitamin-C based on the weight of the composition.

11. The method of claim 1, wherein said malignant tumor is leukemia.

12. The method of claim 1, wherein said malignant tumor is myeloma.
